(19) United States
(12) Patent Application Publication
(10) Pub. No.: US 2014/0012078 A1
(43) Pub. Date: Jan. 9, 2014

(54) Accelorometer Based Endoscopic Light Source Safety System

(71) Applicant: Raymond Coussa, Goleta, CA (US)

(72) Inventor: Raymond Coussa, Goleta, CA (US)

(21) Appl. No.: 13/542,170

(22) Filed: Jul. 5, 2012

(51) Int. Cl. A61B 1/06; A61B 1/04

(52) U.S. Cl. 600/109; 600/117

(57) ABSTRACT

An endoscope, endoscopic system and method that has an automatic-shutoff feature when the endoscope is stationary. The endoscope has a motion sensor and a controller, the motion sensor providing signals to the controller and the controller instructing a light source to reduce its output intensity if the controller does not receive signs from the motion sensor.

### FIELD OF THE INVENTION

The present invention relates to an endoscope and endoscopic system that includes an automatic shutoff feature when the endoscope is stationary. The present invention relates to an automatic shutoff method for an endoscope and endoscopic system where upon non-motion of a stationary endoscope, the light source intensity is automatically dimmed to a safe level or reduced to zero.

### BACKGROUND OF THE INVENTION

The imaging of body surfaces through an endoscope is well known within the medical and veterinarian fields. Endoscopes are used to look in places which typically have no light. It is thus necessary to provide light to an endoscopic scene, meaning that the light which hits an endoscopic camera system (or the user's eye if the user is looking through the eyecup of an optical endoscope) has been pumped into and reflected by the scene.

Typically, this involves inserting an endoscope into a body cavity and directing a high intensity light source output through the endoscope to illuminate body tissue. Traditionally light has been generated by a high-powered light bulb disposed in a table top box and transported to the endoscope through a fiberoptic cable which couples to the endoscope. The endoscope carries the light from the fiberoptic cable to the scene through internal fiberoptics to an image sensor to generate both video and still images of the tissue.

One such approach is described in U.S. Pat. No. 5,162,913 to Chatenever et al., and provides a technique for an automatic adjustment of the exposure of video images detected with a CCD (charge coupled device) image sensor. The contents of U.S. Pat. No. 5,162,913 to Chatenever et al. are herein incorporated by reference in their entirety.

Endoscopes and endoscopic systems serve the dual functions of flooding the scene with light and also collecting that reflected light for imaging purposes. Recently, endoscopes are starting to carry onboard light sources such as LEDs, eliminating the need for the table top illumination box and fiberoptic cable.

Regardless of configuration, a substantial problem with current endoscopic illumination systems is that endoscopic illumination systems sometimes cause fires in the operating room. This happens because surgeons or assistants sometimes set retracted endoscopes down while forgetting to turn down/off the illumination such that the endoscope tip comes too close to surgical drapes and lights them on fire. Various automatic-shutoff schemes centered on image-based motion detection have been conceived in efforts to eliminate this fire hazard, but these are often unreliable.

One such approach to solve this problem is described in U.S. Pat. No. 6,511,422 to Chatenever (hereinafter Chatenever '422). Chatenever '422, herein incorporated by reference in its entirety, describes a method and apparatus where the output from a high intensity light source is controlled so that whenever the output is not directed at tissue (meaning that the endoscope/video camera/light source combination is not currently being used to image body tissue), the light source output intensity is automatically reduced to a safer level. This is done by monitoring the reflected light from tissue and when this reflection indicates that the light source is not directed at tissue, the light intensity is turned down to a safer level. This involves generating a modulation signal and modulating the intensity of the light source output with the modulation signal.

Another such approach to solve this problem is described in U.S. patent application Ser. No. 13/181,350 entitled “Method and apparatus for protection from high intensity light”, filed Jul. 12, 2011 (hereinafter the '350 application). The '350 application involves providing a method and apparatus to upgrade existing and future endoscopic imaging systems with a light source control (“LSC”) feature that solves problems associated with light sources, such as Xenon lights. The contents of the '350 application is herein incorporated by reference in its entirety.

However, none of these prior art references discloses an endoscope that has a feature that reduces and/or shuts off the intensity of the light source based on the lack of motion of an endoscope, i.e., the shutoff feature is applied when the endoscope is not moving or is stationary. None of these prior art references have an automatic-shutoff feature based upon the lack of motion of an endoscope.

Thus, it is desirable to provide a novel and reliable automatic-shutoff feature for endoscopic illumination systems that is based upon the lack of motion of an endoscope.

### SUMMARY OF THE INVENTION

Accordingly, it is an object of the present invention to provide a novel and reliable automatic-shutoff feature for an endoscope and for an endoscopic illumination system based on the lack of motion of an endoscope, i.e., when the endoscope is not moving or is stationary.

It is another object of the present invention to incorporate various sensor elements, such as accelerometers, gyroscopes, magnetometers or similar motion sensors that are typically used for image leveling and endoscopic positioning to detect the non-motion of a stationary endoscope which has been retracted from the surgical cavity and set down.

These and other objects of the invention are achieved by providing an endoscope comprising: a shaft having a proximal end and a distal end; an objective lens disposed in the distal end of the shaft; a light source, said light source providing an output intensity; at least one motion sensor, said at least one motion sensor detecting motion of the endoscope and providing at least one signal if the endoscope is moving; and a controller, said controller processing said at least one signal and instructing said light source to reduce the output intensity if said controller does not receive the at least one signal from the at least one motion sensor.

In certain embodiments, the at least one motion sensor is located within the endoscope shaft. In other embodiments, the at least one motion sensor is located within a camera attached to the endoscope. In other embodiments, the at least one motion sensor is located in a handle attached to the endoscope shaft. In other embodiments, the at least one motion sensor is located remotely from the endoscope and the endoscope shaft.

In certain embodiments, the controller is located within the endoscope shaft. In other embodiments, the controller is located within a camera attached to the endoscope. In other embodiments, the controller is located in a handle attached to the endoscope shaft. In other embodiments, the controller is located remotely from the endoscope and the endoscope shaft.

In certain embodiments, the at least one signal is an electronic signal. In other embodiments, the at least one signal may be a mechanical signal or a signal effectuated by pressure or by an actuator or hydraulics.

In certain embodiments, the at least one motion sensor is continuously detecting motion of the endoscope and continuously sending signals to the controller. In certain embodiments, if the controller fails to receive a signal from the at least one motion sensor, the controller reduces the output intensity of the light source. In certain embodiments, if the endoscope begins to move again and the motion is detected by the at least one motion sensor, the at least one motion sensor sends at least one signal to the controller, and the controller then increases the output intensity of the light source; typically in accordance with auto-exposure capabilities of the controller and/or a computer. The movement of the endoscope is detected by the at least one motion sensor, which is sensitive to movements, such as rotational movement, longitudinal movement along an axis and movement in various planes.

In certain embodiments, the controller instructs said light source to reduce its output intensity to zero (no light being emitted; shut off of the light) when said controller does not receive at least one signal from the at least one motion sensor.

In certain embodiments, determining whether said endoscope is moving comprises testing whether readings from said at least one motion sensor are above or below a reference threshold. In certain embodiments, the reference threshold can be adjusted based upon the specificity of a surgeon.

In certain embodiments, the endoscope includes software executing on the controller. In certain embodiments, said controller decrements the intensity of the light source output via a plurality of steps.

In certain embodiments, the light source is a Xenon light. In certain embodiments, the light source is a LED. In certain embodiments, the light source intensity is decremented by a predetermined percentage of the maximum light source output intensity.

In certain embodiments, the light source is disposed in said distal end of said shaft. In certain embodiments, the light source is disposed remotely from said endoscope.

In certain embodiments, the controller is disposed in said endoscope. In certain embodiments, the controller is disposed remotely from said endoscope.

In certain embodiments, the at least one motion sensor is selected from the group consisting of an accelerometer, gyroscope, and magnetometer.

In certain embodiments, the at least one motion sensor is used to detect the motion of the endoscope after the endoscope has been retracted from a surgical cavity and set down. In certain embodiments, the endoscope is set down on a table, such as in an operating room.

In certain embodiments, the endoscope has a handle. In certain embodiments, the at least one motion sensor is disposed in the handle.

In certain embodiments, the objective lens is part of an imaging system. In certain embodiments, the light source output intensity is reduced automatically to a safe level if the endoscope is not moving. In certain embodiments, the light source output intensity is reduced to zero (shutoff) automatically if the endoscope is not moving.

Other objects of the invention are achieved by providing a surgical system comprising: an examining instrument having a shaft having a proximal end and a distal end, an objective lens disposed in the distal end of the shaft, a light source, said light source providing an output intensity, at least one motion sensor, said at least one motion sensor detecting motion of the endoscope and providing at least one signal if the endoscope is moving, and a controller, said controller processing said at least one signal and instructing said light source to reduce the output intensity of said light source if said controller does not receive at least one signal from the at least one motion sensor; a display to display images from the electronic imaging system; and a computer, the computer including said software module.

In certain embodiments, the at least one motion sensor is located within the endoscope shaft. In other embodiments, the at least one motion sensor is located within a camera attached to the endoscope. In other embodiments, the at least one motion sensor is located in a handle attached to the endoscope shaft. In other embodiments, the at least one motion sensor is located remotely from the endoscope and the endoscope shaft.

In certain embodiments, the controller is located within the endoscope shaft. In other embodiments, the controller is located within a camera attached to the endoscope. In other embodiments, the controller is located in a handle attached to the endoscope shaft. In other embodiments, the controller is located remotely from the endoscope and the endoscope shaft.

In certain embodiments, the examining instrument is selected from a group consisting of an endoscope, laryngoscope, bronchoscope, fiberscope, duodenoscope, gastroscope, flexible endoscope, arthroscope, cystoscope, laparoscope, anoscope, and sigmoidoscope.

In certain embodiments, the controller instructs said light source to reduce its output intensity to zero when said controller does not receive at least one signal from the at least one motion sensor.

In certain embodiments, determining whether said examining instrument is moving comprises testing whether readings from said at least one motion sensor is above or below a reference threshold.

In certain embodiments, the system further comprises software executing on the controller.

In certain embodiments, the controller decrements the intensity of the light source output via a plurality of steps.

In certain embodiments, the at least one motion sensor is selected from the group consisting of an accelerometer, gyroscope, and magnetometer.

Other objects of the invention are achieved by providing a method for automatically reducing the intensity of an endoscope light source comprising: providing an endoscope having a light source and at least one motion sensor; automatically detecting motion of the endoscope and providing at least one signal if the endoscope is moving; sending the at least one signal from the at least one motion sensor to a controller; processing the at least one signal; and reducing the output intensity of the light source when the controller does not receive at least one signal from the at least one motion sensor.

In certain embodiments, the output intensity of the light is reduced to zero when said controller does not receive at least one signal from the at least one motion sensor.

In certain embodiments, the at least one motion sensor continuously detects motion of the endoscope. In certain embodiments, the at least one motion sensor detects motion of the endoscope every few seconds.

Other objects of the invention are achieved by providing an endoscope comprising a shaft having a proximal end and a distal end; an objective lens disposed in the distal end of the shaft; a light source, said light source providing an output intensity; at least one motion sensor, said at least one motion sensor continuously detecting motion of the endoscope and continuously providing signals if the endoscope is moving; and a controller, said controller continuously processing said signals and instructing said light source to reduce the output intensity if said controller does not continuously receive the signals from the at least one motion sensor.

In certain embodiments, the at least one motion sensor is located within the endoscope shaft. In other embodiments, the at least one motion sensor is located within a camera attached to the endoscope. In other embodiments, the at least one motion sensor is located in a handle attached to the endoscope shaft. In other embodiments, the at least one motion sensor is located remotely from the endoscope and the endoscope shaft.

In certain embodiments, the controller is located within the endoscope shaft. In other embodiments, the controller is located within a camera attached to the endoscope. In other embodiments, the controller is located in a handle attached to the endoscope shaft. In other embodiments, the controller is located remotely from the endoscope and the endoscope shaft.

In certain embodiments, the at least one motion sensor detects motion of the endoscope every few seconds. In other embodiments, the at least one motion sensor detects motion of the endoscope every second or in fractions of a second.

In certain embodiments, the at least one motion sensor sends a signal to the controller every few seconds. In certain embodiments, the at least one motion sensor sends a signal to the controller every second or in fractions of a second.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a perspective view of an endoscope of an embodiment of the invention;

FIG. 2 is a detailed side view of the endoscope of FIG. 1; and

FIG. 3 is a flowchart of an embodiment of the invention.

### DETAILED DESCRIPTION OF THE INVENTION

The following detailed description illustrates the invention by way of example, not by way of limitation of the principles of the invention. This description will enable one skilled in the art to make and use the invention, and describes several embodiments, adaptations, variations, alternatives and uses of the invention, including what is presently believed to be the best mode of carrying out the invention.

The present invention is directed to a novel, unique and improved endoscope and endoscopic imaging system having at least one motion sensor used to detect the non-motion of a stationary endoscope which has been retracted from a surgical cavity and set down. The present invention is directed to an endoscope that has a feature that reduces and/or shuts off the intensity of the light source based on the lack of motion of an endoscope, i.e., when the endoscope is not moving or is stationary.

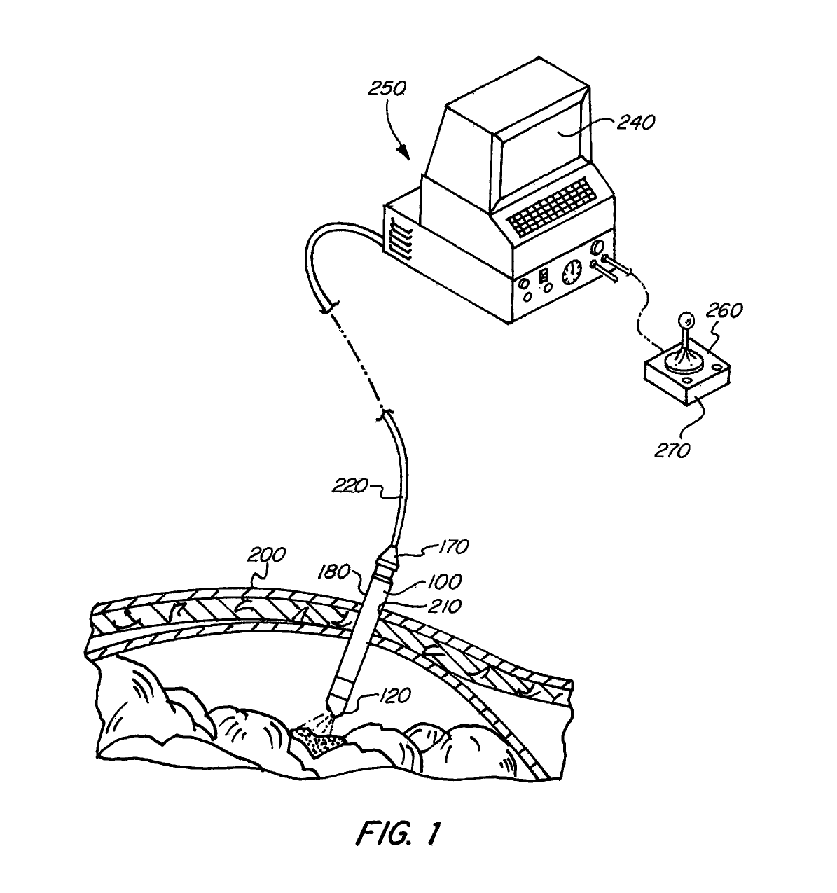

FIG. 1 shows a view of the endoscope of an embodiment of the invention where the endoscope 100 is being used through the skin 200 of a patient. In FIG. 1, endoscope 100 is shown being able to pass through hole 210 in the skin of a person. The hole 210 may preferably have a diameter of approximately 15 mm or even less. Endoscope 100 may also be able to pass through a trocar.

Endoscope 100 is shown connected via cable 220 to a display 240, which may or may not be part of a computer 250, and which is controlled by keypad or joystick 260/270. The joystick 260/270 may provide a control of the endoscope 100. In certain embodiments, the joystick 260/270 may be replaced by a keypad, keyboard, or other input element.

Endoscope 100 is also shown having a distal tip 120 where the light source is located. The light source may also be present in the proximal end of the endoscope (not shown).

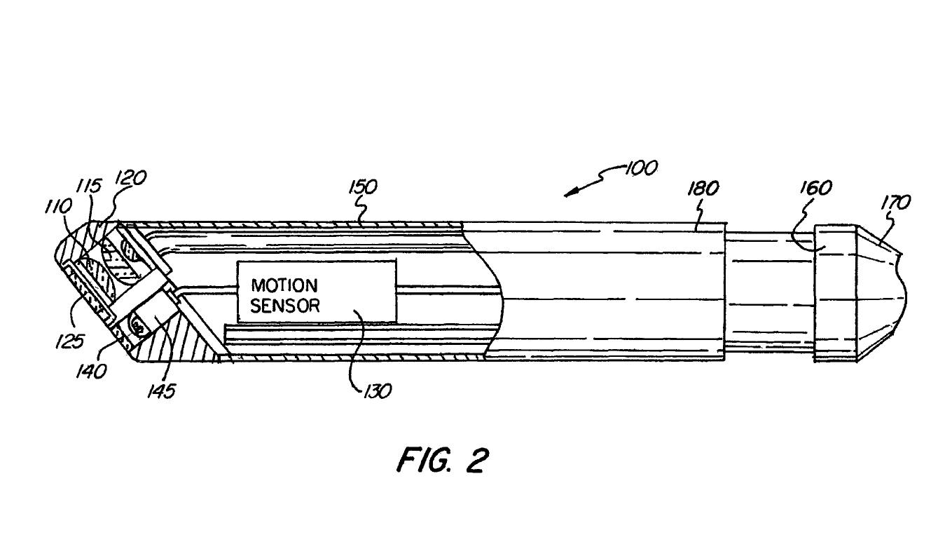

FIG. 2 shows a detailed side view of the endoscope of FIG. 1. FIG. 2 has a motion sensor 130, which may be an accelerometer, gyroscope, and magnetometer or a combination of these elements.

Furthermore, there may be additional motion sensors (which are not shown in FIG. 2). These motion sensors may serve the purposes of image leveling and endoscopic positioning, and may also serve a secondary purpose of detecting the motion of an endoscope which has been retracted from the surgical cavity and set down.

The motion sensor(s) may be used to detect if the endoscope is rotating. Rotational movement and axial displacement are considered movement of the endoscope.

Since the motion sensors are conveniently already onboard the endoscope, the motion sensors can serve the secondary purpose of signaling and instructing the illumination source to dim or turn off whenever the endoscope is stationary. Unless the endoscope is mounted on an arm, which is infrequent, it will never be completely stationary whether it is in the operator's hand, and so there is very little risk of false alarms.

FIG. 2 also shows the other internal components of the endoscope of FIG. 1. FIG. 2 may include magnets (not shown) and may include various camera electronic elements such as an illuminator 110/115 (light source) and an objective lens/imager 145. Lens cover 125 and lens cover 140 are shown covering the illuminator 110/115 and objective lens/imager 145 respectively to form a sealed window assembly. These elements are shown located in the distal end 120 of the endoscope. The endoscope can be sterilized in an autoclave.

A controller (not shown) may be located in the endoscope or may be disposed remotely from the endoscope, and may be located in the computer 250, for example. The endoscope 100 is shown having a shaft 150 with proximal end 160 and distal tip (distal end) 120.

The endoscope 100 may have a circuitry, such as an integrated circuit (not shown) that can include software for processing images.

In certain embodiments, the illuminator 110/115 may be a Xenon light. In certain embodiments, endoscope 100 may include a handle portion 180. A surgeon can move and rotate endoscope 100 via handle portion 180. In certain embodiments, the motion sensors may be located in the handle portion 180.

In certain embodiments, the illuminator may include a wide-angle lens or lenses. In certain embodiments, the components disposed within distal end 120 may include a ⅙″ CCD sensor, a white light LED, a temperature sensor, an IDROM (identification read only memory), an I2C bus, and video and timing interface electronics.

In certain embodiments, the endoscope is intended to survive an autoclave sterilization cycles using an included sterilization carrier.

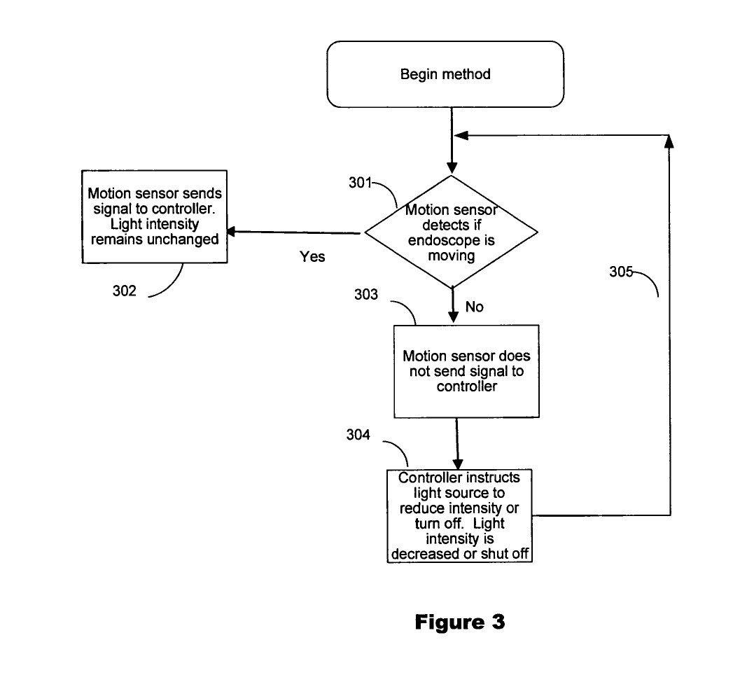

FIG. 3 is a flowchart of an embodiment of the invention. FIG. 3 involves the motion sensor detecting if the endoscope is moving at step 301. If yes, then the motion sensor sends a signal 302 to the controller. The controller processes the signal and the light intensity of the light source remains unchanged.

If the motion detector fails to detect motion (i.e., the endoscope is stationary or has non-motion), the motion detector fails to send a signal 303 to the controller. If the controller, which is programmed to check for a signal from the motion sensor every few seconds, does not receive a signal from the motion sensor, will instruct the light source to reduce its intensity or turn off (304). The controller may send a signal to the light source to do so. In other embodiments, the failure of the controller to send a signal to the light source may cause it to turn off or reduce its light output intensity level.

The method may be repeated continuously as the motion sensor may check for motion every few seconds.

While the invention has been specifically described in connection with certain specific embodiments thereof, it is to be understood that this is by way of illustration and not of limitation and that various changes and modifications in form and details may be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

What is claimed is:

1. An endoscope comprising:
a shaft having a proximal end and a distal end;
an objective lens disposed in the distal end of the shaft;
a light source, said light source providing an output intensity;
at least one motion sensor, said at least one motion sensor detecting motion of the endoscope and providing at least one signal if the endoscope is moving; and
a controller, said controller processing said at least one signal and instructing said light source to reduce the output intensity if said controller does not receive the at least one signal from the at least one motion sensor.

2. The endoscope of claim 1, wherein said controller instructs said light source to reduce its output intensity to zero when said controller does not receive at least one signal from the at least one motion sensor.

3. The endoscope of claim 1, wherein determining whether said endoscope is moving comprises testing whether readings from said at least one motion sensor are above or below a reference threshold.

4. The endoscope of claim 1, further comprising software executing on the controller.

5. The endoscope of claim 1, wherein said controller decrements the intensity of the light source output via a plurality of steps.

6. The endoscope of claim 1, wherein said light source is a Xenon light.

7. The endoscope of claim 1, wherein said light source intensity is decremented by a predetermined percentage of the maximum light source output intensity.

8. The endoscope of claim 1, wherein said light source is disposed in said distal end of said shaft.

9. The endoscope of claim 1, wherein said light source is disposed remotely from said endoscope.

10. The endoscope of claim 1, wherein said controller is disposed in said endoscope.

11. The endoscope of claim 1, wherein said controller is disposed remotely from said endoscope.

12. The endoscope of claim 1, wherein said at least one motion sensor is selected from the group consisting of an accelerometer, gyroscope, and magnetometer.

13. The endoscope of claim 1, wherein said at least one motion sensor is used to detect the motion of the endoscope after the endoscope has been retracted from a surgical cavity and set down.

14. The endoscope of claim 1, further comprising a handle.

15. The endoscope of claim 14, wherein said at least one motion sensor is disposed in the handle.

16. The endoscope of claim 1, wherein the objective lens is part of an imaging system.

17. The endoscope of claim 1, wherein said light source output intensity is reduced automatically if the endoscope is not moving.

18. A surgical system comprising:
an examining instrument having
a shaft having a proximal end and a distal end,
an objective lens disposed in the distal end of the shaft,
a light source, said light source providing an output intensity,
at least one motion sensor, said at least one motion sensor detecting motion of the endoscope and providing at least one signal if the endoscope is moving, and
a controller, said controller processing said at least one signal and instructing said light source to reduce the output intensity of said light source if said controller does not receive at least one signal from the at least one motion sensor;
a display to display images from the electronic imaging system; and
a computer, the computer including a software module.

19. The surgical system of claim 18, wherein the examining instrument is selected from a group consisting of an endoscope, laryngoscope, bronchoscope, fiberscope, duodenoscope, gastroscope, flexible endoscope, arthroscope, cystoscope, laparoscope, anoscope, and sigmoidoscope.

20. The surgical system of claim 18, wherein said controller instructs said light source to reduce its output intensity to zero when said controller does not receive at least one signal from the at least one motion sensor.

21. The surgical system of claim 18, wherein determining whether said examining instrument is moving comprises testing whether readings from said at least one motion sensor is above or below a reference threshold.

22. The surgical system of claim 18, further comprising software executing on the controller.

23. The surgical system of claim 18, wherein said controller decrements the intensity of the light source output via a plurality of steps.

24. The surgical system of claim 18, wherein said at least one motion sensor is selected from the group consisting of an accelerometer, gyroscope, and magnetometer.

25. A method for automatically reducing the intensity of an endoscope light source comprising:
providing an endoscope having a light source and at least one motion sensor;
automatically detecting motion of the endoscope and providing at least one signal if the endoscope is moving;
sending the at least one signal from the at least one motion sensor to a controller;
processing the at least one signal; and
reducing the output intensity of the light source when the controller does not receive at least one signal from the at least one motion sensor.

26. The method of claim 25, wherein the output intensity of the light is reduced to zero when said controller does not receive at least one signal from the at least one motion sensor.
